(19) United States
(12) Patent Application Publication
(10) Pub. No.: US 2010/0291166 A1
(43) Pub. Date: Nov. 18, 2010

(54) USE OF NITROGEN-DOPED TITANIUM OXIDE NANOPARTICLES AS AGENTS FOR PROTECTING AGAINST ULTRAVIOLET RADIATION

(75) Inventors: Véronique Guyot-Ferreol, Paris (FR); Jean-Francois Tranchant, Marigny Les Usages (FR); Hicham Maskrot, Antony (FR)

Correspondence Address: MERCHANT & GOULD PC, P.O. BOX 2903, MINNEAPOLIS, MN, 55402-0903, US

(73) Assignee: LVMH Recherche, Saint Jean de Braye (FR)

(21) Appl. No.: 12/676,539

(22) PCT Filed: Sep. 12, 2008
(86) PCT No.: PCT/FR2008/051628

§ 371 Date: Aug. 3, 2010

(30) Foreign Application Priority Data
Sep. 13, 2007 (FR) 0757550

(51) Int. Cl. A61K 8/02; A61K 8/18; A61Q 17/04

(52) U.S. Cl. 424/401; 424/59

(57) ABSTRACT

A nanometric material includes nitrogen-doped titanium oxides, which is obtained by injection of a titanium oxide precursor in the liquid or gaseous form and of a gaseous nitrogenous compound into a laser pyrolysis reactor. The material is used as cosmetic agent for protecting against ultraviolet radiation, thereby improving protection against UV radiation and in particular against UV-A radiation.

The invention relates to the use of nitrogen-doped titanium oxide nanoparticles as agent for protecting against ultraviolet radiation.

A more particular subject matter of the invention is the use of a nanometric material based on titanium oxide, obtained using a laser pyrolysis device, as material for protecting from ultraviolet radiation. The invention also relates to the cosmetic compositions including said nanometric material which are intended for the protection of the skin with regard to ultraviolet (UV) radiation.

### STATE OF THE ART

Commercial suncreams have the aim of protecting the skin from UV-B radiation (radiation of between 290 nm and 320 nm) and UV-A radiation (320 nm to 400 nm) by virtue of the organic and/or inorganic sunscreens present therein, said radiation being responsible for erythema and skin burns and also for accelerated aging of the skin, indeed even for some cancers.

Depending on their wavelength, these types of radiation penetrate deep, reach the dermis and are capable of bringing about phototoxic or photoallergic reactions, in particular in the case of light-skinned people. In addition, UV-A radiation weakens the structural proteins of the skin, in particular collagen and elastin fibers, thus reducing the tone and elasticity of the skin, and results in the appearance of wrinkles for skin continually exposed to solar radiation. Finally, UV-A radiation is also the cause of melanomas by their mutagenic action.

A recent realization recommends the use of high protection factor (SPF) suncreams or sunscreens which absorb the entire UV/visible spectrum (total screen). The most effective creams are composed of opaque inorganic oxides and more particularly of titanium dioxide (TiO₂) and of zinc oxide (ZnO), in combination with organic screening agents, such as, for example, paraaminobenzoic acid, benzophenone derivatives and camphor derivatives, for adjusting and increasing the protection factor (SPF) of the product.

However, effective organic screening agents corresponding to the large UV wavelengths ranging from 370 nm to 400 nm do not exist. Furthermore, it has been reported that some of these screening agents exhibit in vitro estrogenic activities, that is to say that they behave like female hormones (M. Schlumpf et al., SOFW Journal, 127(7), (2001), pages 10-15, and Environ. Health Perspec., 109(11), (2001), page A517). Today, use of suncreams with an increasingly high protection factor results in increased absorption of the organic screening agents by the skin. Thus, some benzophenone derivatives have been detected in human urine 4 hours after application to the skin of a suncream in which it was present. Some screening agents also become pollutants and are encountered in swimming water, fish and human milk.

It should also be noted that the current products with a high photoprotective power are composed of organic sunscreens and of mixtures of metal oxide particles, such as TiO₂ particles. Titanium dioxide is a semiconductor material which, when it is irradiated by UV radiation, can induce photocatalytic phenomena.

In order to overcome this problem, the TiO₂ particles thus have to be photostabilized by surface treatment, as described, for example, in patent EP-B-0 461 130, where TiO₂ nanoparticles were treated with phosphate anions.

More generally, cosmetic products are found to include TiO₂ nanoparticles, the surface of which has been coated with alumina or, preferably, with silica (see patent applications EP-A-0 518 772 and EP-A-0 518 773).

However, these systems do not solve the problem of organic screening agents, which can penetrate through the skin (M. Schlumpf et al., ibid.).

The proposal has been made, in order to find a solution to this problem, to encapsulate organic sunscreens in silica particles, as described, for example, in patent applications WO 2003/011239 and WO 2002/078665 and the publications by N. Lapidot et al., Journal of Sol-Gel Science and Technology, 26 (1/2/3), (2003), pages 67-72, by F. Pflueker et al., SOFW Journal, 128(6), (2002), pages 24-26, and by C. Anselmi et al., International Journal of Pharmaceutics, 242(1-2), (2002), pages 207-211.

Yet other solutions have been proposed in order to overcome the disadvantage of the inadequate absorption of the UV-A radiation, such as, for example, by combining a zirconium compound with the screening agents (cf. FR 2 799 120).

However, there are numerous disadvantages to all these approaches, among which may be mentioned, on the one hand, treatments which are expensive and difficult to implement and, on the other hand, inadequate absorption of the UV-A radiation since the organic screening agents and the silica do not absorb radiation with a wavelength of greater than 360 nm.

There thus currently remains a need for materials which make possible effective protection over a broader extent of the UV spectrum and which are effective in particular for protection against UV-A radiation, while limiting the amount of organic screening agents capable of penetrating through the skin.

### AIMS AND OBJECTS OF THE INVENTION

Thus, a first object of the present invention is to provide novel materials for protecting against UV radiation which do not exhibit the abovementioned disadvantages.

In particular, an object of the present invention is to provide a material capable of absorbing most of the radiation of the UV-A and UV-B spectrum, said material being only slightly absorbed or not absorbed at all by the skin.

Another object is to provide a material capable of absorbing the greatest possible part of the radiation of the UV-A and UV-B spectrum and which induces nothing or little in the way of photocatalytic phenomena.

Another object is to provide a material which, in addition to the absorption over the whole of the radiation of the UV spectrum, absorbs significantly in the UV-A radiation. Such a property is particularly advantageous for photoinduced aging phenomena.

Another object is to prevent or radically reduce the penetration of the organic screening agents through the skin.

Yet another object is to provide a material which is harmless or virtually harmless to the environment.

The inventors have now discovered, surprisingly, that the objects described above are achieved, in whole or in part, by virtue of the use of nanometric materials based on titanium oxide according to the invention, which materials will be described in detail below.

### DETAILED DESCRIPTION OF THE INVENTION

According to a first aspect, the present invention relates to the use of a nanometric material based on nitrogen-doped titanium oxides as cosmetic agent for protecting against ultraviolet radiation.

According to a specific embodiment, the nanometric material based on nitrogen-doped titanium oxide is obtained by injection of a titanium oxide precursor in the liquid or gaseous form and of a gaseous nitrogenous compound into a laser pyrolysis reactor.

The invention is not limited to this type of process in obtaining the nanometric material based on nitrogen-doped titanium oxide. A person skilled in the art knows other methods for manufacturing this type of material.

The invention thus covers all the uses of any nanometric material based on nitrogen-doped titanium oxide which can be manufactured as cosmetic agent for protecting against ultraviolet radiation.

According to another specific embodiment of the invention, the nanometric material based on nitrogen-doped titanium oxide comprises at least 0.4% of nitrogen atoms, with respect to the total atomic composition of the nanometric material, and between 0 and 40% by weight of carbon, with respect to the total weight of the nanometric material or of the nanoparticles.

According to a specific embodiment, this material based on nitrogen-doped titanium oxide comprises from 0.5 to 10% of nitrogen atoms.

According to a specific embodiment of the invention, the precursor comprises or is essentially composed of titanium tetraisopropoxide (TTIP) or titanium tetrachloride (TiCl₄) or their mixture. When TiCl₄ is used, oxygen is added by injecting an oxygen (O₂) stream.

According to another specific embodiment of the invention, the nitrogenous compound comprises or is essentially composed of ammonia in the liquid form or of gaseous ammonia NH₃ or of monomethylamine (CH₃NH₂) or their mixture.

According to a specific alternative embodiment, the flow rate of the nitrogenous compound is between 10 and 2000 cm³ per minute.

According to another specific embodiment, the precursor in the gaseous state exiting from the evaporator is entrained by a neutral gas into said reactor, in particular at a flow rate of 200 cm³ per minute and 4000 cm³ per minute, preferably between 500 and 2000 cm³ per minute.

According to a specific alternative embodiment, the carrier gas comprises a sensitizing additive having the role of increasing the absorption of laser energy.

According to another specific embodiment, the flow rate of the precursor is less than or equal to 1000 g per hour, thus continuously producing TiO₂ nanoparticles which are devoid of carbon or which comprise between 0 and 40% by weight of carbon, with respect to the weight of the nanometric material.

According to a specific alternative embodiment, the flow rate of the precursor, in particular in the liquid form, is of the order of 100 g per hour, thus producing, for example, more than 20 g of TiO₂ particles comprising at least 0.4% of nitrogen atoms which are devoid of carbon or which comprise between 0 and 40% by weight of carbon, with respect to the total weight of the nanoparticles.

According to yet another specific embodiment, when the material obtained after the laser pyrolysis comprises carbon, this material is subjected to an additional heat treatment which removes the carbon in order to produce TiO₂ nanoparticles.

According to a specific embodiment, the laser power is between 500 and 2500 watts.

According to yet another specific embodiment, said material has undergone a surface treatment chosen from:
a) a photostabilization treatment, for example with phosphate anions; and/or
b) at least one treatment for coating with at least one coating layer, for example at least one alumina and/or silica coating layer.

These photostabilization or coating treatments are well known to a person skilled in the art and have also been mentioned in the preceding part discussing the state of the art, in particular EP-B-0 461 130, where TiO₂ nanoparticles were treated with phosphate anions, and more generally TiO₂ nanoparticles having a surface coating with alumina or preferably silica (see patent applications EP-A-0 518 772 and EP-A-0 518 773).

According to a second aspect, the present invention also relates to a cosmetic composition comprising an effective amount of at least one nanometric material as defined above or as described in the following description comprising exemplary embodiments of the invention as cosmetic agent for protecting against ultraviolet radiation, alone or in combination with one or more other organic and/or inorganic sunscreens, in a medium acceptable from the cosmetic viewpoint.

According to a specific embodiment, this cosmetic composition comprises from 0.1 to 50% by weight and preferably from 1 to 15% by weight of said nanomaterial, with respect to the total weight of the composition.

According to other specific embodiments, this composition is characterized in that it is provided in the cream, oil, gel, spray, lotion or powder form.

According to yet another specific embodiment, this cosmetic composition is characterized in that said material has undergone a surface treatment chosen from:
a) a photostabilization treatment, for example with phosphate anions; and/or
b) at least one treatment for coating with at least one coating layer, for example at least one alumina and/or silica coating layer.

In the context of the present description and of the claims, the term “nanometric material” is understood to mean a nanometric material or nanoparticles having a mean diameter of between 2 nm and 100 nm, advantageously between 6 nm and 30 nm, in a nonagglomerated elementary form. The agglomerated forms are also included in the invention.

In the context of the present description and of the claims, the terms “nanometric material” and “nanoparticles” are used indifferently and have the same meaning.

Furthermore, nanoparticles having a mean diameter of less than 50 nm are preferred as, for a mean diameter of greater than 50 nm, an undesirable visual effect, in particular an effect of whitening of the skin, may be produced in some cases, for example in the case where the metal chosen is titanium, in the oxidized form TiO₂.

Also, in the context of the present description and of the claims, “organic sunscreen” is understood to mean any organic compound which absorbs UV radiation in the wavelength range extending generally from 250 nm to 400 nm, without this, however, constituting a limit.

Other aims, characteristics and advantages of the invention will become clearly apparent from the explanatory description which will follow, made with reference to several exemplary embodiments of the invention given simply by way of illustration and which should thus in no way limit the scope of the invention. In the examples, the temperature is in degrees Celsius, the pressure is atmospheric pressure and the percentages are given by weight, unless otherwise indicated.

### DESCRIPTION OF THE FIGURES

FIG. 1 represents a block diagram of a laser pyrolysis device which can be used to prepare the materials based on titanium oxides, with addition of an additive comprising a nitrogenous compound in order to carry out doping with nitrogen.

FIG. 2 represents a curve of the results obtained with a material prepared in example 1A, known as TiOCN 16 NR, comprising nitrogen, in comparison with the titanium dioxide material of commerce known under the trade name M160, sold by Kemira, with, on the abscissa, the wavelength of the ultraviolet radiation, expressed in nanometers, and, on the ordinate, the transmittance, expressed in arbitrary units.

FIG. 3 represents a curve of the results obtained with a material prepared in example 1D, known as TiOCN 182 NR, comprising nitrogen, in comparison with the titanium dioxide material of commerce known under the trade name M160, sold by Kemira, with, on the abscissa, the wavelength of the ultraviolet radiation, expressed in nanometers, and, on the ordinate, the transmittance, expressed in arbitrary units.

FIG. 4 represents a curve of the results obtained with a material prepared in example 1B, known as TiOCN 123 NR, comprising nitrogen, in comparison with the titanium dioxide material of commerce known under the trade name M160, sold by Kemira, with, on the abscissa, the wavelength of the ultraviolet radiation, expressed in nanometers, and, on the ordinate, the transmittance, expressed in arbitrary units.

FIG. 5 represents a curve of the results obtained with a material prepared in example 1C, known as TiOCN 127 NR, comprising nitrogen, in comparison with the titanium dioxide material of commerce known under the trade name M160, sold by Kemira, with, on the abscissa, the wavelength of the ultraviolet radiation, expressed in nanometers, and, on the ordinate, the transmittance, expressed in arbitrary units.

FIG. 6 represents a curve of the results obtained with a material prepared in example 1E, known as TiON 16 R, which is devoid of carbon after an “annealing” heat treatment lasting three hours, in comparison with the titanium dioxide material of commerce known under the trade name M160 sold by Kemira, with, on the abscissa, the wavelength of the ultraviolet radiation, expressed in nanometers, and, on the ordinate, the transmittance, expressed in arbitrary units.

### EXAMPLE 1

### Preparation of Nanometric Material According to the Invention

### Description of the Process

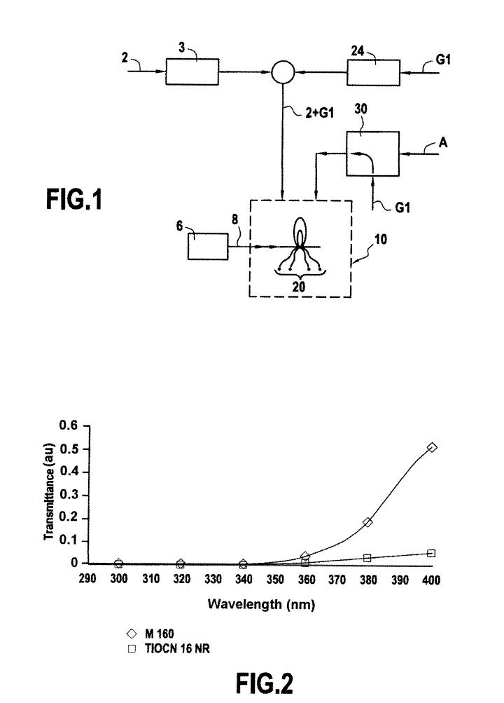

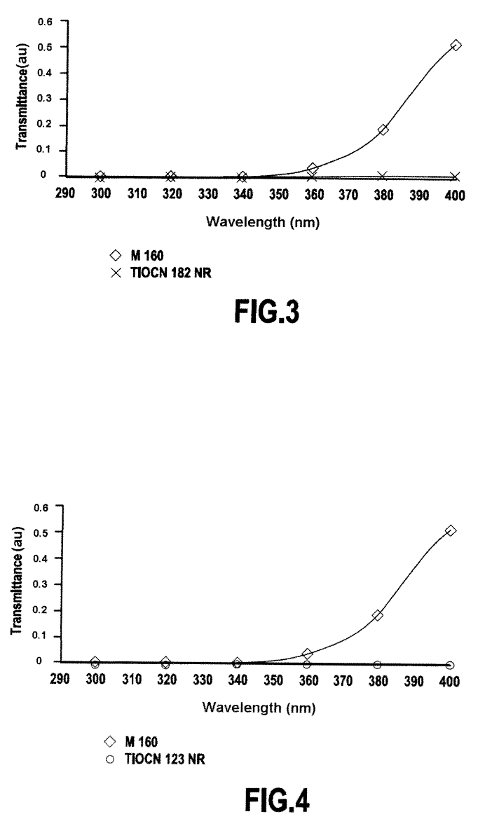

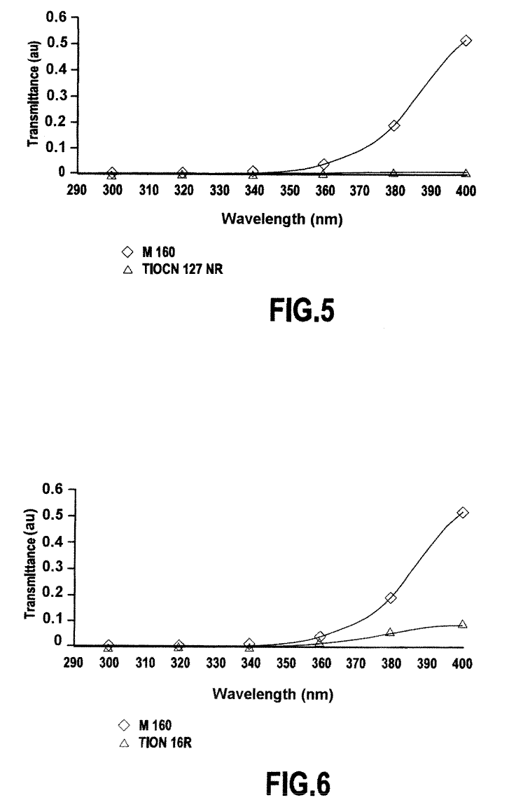

With reference to FIG. 1, in the laser pyrolysis process applied to the synthesis of nitrogen-doped titanium dioxide nanoparticles, a titanium dioxide precursor 2, for example titanium tetraisopropoxide (TTIP), interacts with a laser beam 8 in a pyrolysis reactor 10 to produce nanoparticles 20.

According to the invention, the TTIP is injected into the reactor 10 continuously (non-pulsed mode).

Preferably, the TTIP flow rate is substantially continuous and controlled by a mass flow rate controller 3 and more particularly the TTIP is in the liquid phase but can also be converted to the vapor phase in an evaporator 4 (not represented) before being injected into the reactor 10; be that as it may, the TTIP flow rate is controlled in the liquid phase by the abovementioned mass flow rate controller 3.

The mass flow rate of the TTIP in the liquid form for the laser pyrolysis reaction can range up to 1000 grams per hour. For a flow rate in the liquid form of the order of 100 grams per hour TTIP, the pyrolysis can produce more than 20 grams of TiOCN nanoparticles per hour and in particular more than 25 grams per hour.

Advantageously, the TTIP in the liquid phase or in the vapor phase can be carried by a gas G1 into the reactor 10, advantageously via a flow rate controller 24. This gas is a neutral gas, argon or helium, but it can also comprise a sensitizing additive A, for example comprising a hydrocarbon compound, such as ethylene, in order to transfer, if need be, energy to the TTIP during the pyrolysis. This additive A can be added in the liquid or gaseous form.

In order to carry out doping with nitrogen, a nitrogenous additive A comprising a gaseous nitrogenous compound, for example liquid ammonia or gaseous ammonia, NH₃, will be injected simultaneously with the precursor, independently, as represented, or as a mixture with the latter. In this case, the liquid or gaseous nitrogenous additive A can either be mixed with the carrier gas G1 in a mixer 30 or this gaseous nitrogenous additive can essentially constitute the carrier gas. Thus, the nitrogenous additive is mixed and is combined with the precursor 2 to form the TiOCN nanoparticles in the reactor 10. Preference is given, as nitrogenous additive, to liquid ammonia or better still to gaseous ammonia, NH₃.

According to another specific embodiment, nanoparticles of formula TiON, that is to say devoid of carbon, are obtained. In this case, a “thermal annealing” heat treatment will additionally be carried out at a low temperature, generally not greater than 400° C., typically between 200 and 400° C., in ambient atmosphere, on the nanoparticles 20 obtained until the carbon has disappeared. It also transpired that, for an annealing temperature of greater than 400° C., it is possible for the crystalline structure of the particles to be modified.

Examples 1A to 1D of TiOCN nanomaterials (without annealing) obtained according to the process described above as a function of the operating conditions are given in TABLES I (injection of the precursor in the liquid form) and II (injection of the precursor in the gaseous state) as a function of the operating conditions

TABLE I
Nanomaterials of formula TiOCN by liquid injection

| | | Carrier | NH₃ flow | Laser | Level of nitrogen | Level | UV |
|---|---|---|---|---|---|---|---|
| Liquid | Carrier | gas flow rate | rate | power | (% of nitrogen | of | attenuation |
| injection | gas | (cm³/min) | (g/h) | (W) | atoms) | carbon | value |
| Ex. 1A | Helium | 2000 | 400 | 670 | 1.1% | 1.4 | 1000 |
| TiOCN | | | | | | | |
| 16NR | | | | | | | |
| Ex. 1B | Argon | 500 | <20 | 1060 | 3.2% | 10.8% | 4600 |
| TiOCN | | | | | | | |
| 123 NR | | | | | | | |
| Ex. 1C | Nitrogen | 750 | 100 | 1060 | 8% | 3.8% | 4000 |
| TiOCN | | | | | | | |
| 127 NR | | | | | | | |

TABLE II
Nanomaterials of formula TiOCN by gaseous injection

| | | Carrier | NH₃ flow | Laser | Level | Level | UV |
|---|---|---|---|---|---|---|---|
| Vapor | Carrier | gas flow rate | rate | power | of | of | attenuation |
| injection | gas | (cm³/min) | (g/h) | (W) | nitrogen | carbon | value |
| Ex. 1D | Argon | 500 | 100 | 2360 | ND | ND | 2400 |
| TiOCN | | | | | | | |
| 182 NR | | | | | | | |

ND: not determined

Examples 1E to 1F of TiON nanomaterials (with annealing treatment) obtained according to the process described above as a function of the operating conditions are given in TABLE III (injection of the precursor in the liquid form)

TABLE III
Nanomaterials of formula TiON by liquid injection

| | | Carrier | NH₃ flow | Laser | | Level of nitrogen | UV |
|---|---|---|---|---|---|---|---|
| Liquid | Carrier | gas flow rate | rate | power | Annealing | (% of nitrogen | attenuation |
| injection | gas | (cm³/min) | (g/h) | (W) | time/T | atoms) | value |
| Ex. 1E | Helium | 2000 | 400 | 670 | 3 H/ | 0.4% | 655 |
| TiON | | | | | 400° C. | | |
| 16 R | | | | | | | |

The determination of the attenuation of the light intensity transmitted in the range of the ultraviolet radiation which is reported in tables Ito III was measured by the following method of measuring the UV attenuation.

Method of Measuring the UV Attenuation 0.5 g of test powder+2.0 g of castor oil are milled using a disk mill at the rate of 2 times 100 revolutions. The milled product is subsequently dispersed in a collodion (15% nitrocellulose-42.5% ethyl acetate/42.5% butyl acetate) using ultrasound for 15 min.

The dispersion is subsequently spread, using an automatic film draw, over PMMA plates which are transparent to UV radiation. The thickness of the wet film is 300 μm.

A film composed of collodion and of castor oil is prepared in order to act as reference in the spectrophotometric measurement.

When the films are dry, the blank is prepared using the reference plate. The measurements are then carried out on a Scientec OL754 spectrophotometer equipped with a 75W xenon arc lamp. The intensity of the light transmitted through the film between 290 nm and 400 nm is then recovered.

An algorithm makes it possible, from this curve I=f(λ) to obtain a numerical value which, in the case where a suncream is spread over PMMA plates, corresponds to the in vitro sun protection coefficient.

The results in the tables also form the subject of the curves reported in the appended single FIGURE.

### CONCLUSIONS

It is observed that the nanometric product of the invention makes it possible to provide effective protection against ultraviolet radiation and in particular effective protection against UV-A rays which is significantly and completely unexpectedly improved, with respect to the nanometric product of commerce UV-Titan M160.

Various examples of formulations of cosmetic products according to the invention, given simply by way of illustration, will be described below.

In the formulations below, the amounts are given as percentages by weight.

### EXAMPLE 2

### Foundation

| | |
|---|---|
| Polyether-modified polysiloxane | 3.00 |
| Sorbitan, 9-octadecenoate (2:3) | 1.00 |
| Isododecane | 8.00 |
| Pentacyclomethicone | 18.51 |
| Inorganic pigments | 10.00 |
| Phenoxyethanol | 0.30 |
| Bentone gel VS 5PC V | 3.00 |
| Water | 44.62 |
| Tetrasodium EDTA | 0.10 |
| Sodium chloride | 2.00 |
| 1,3-Butylene glycol | 5.00 |
| Methyl para-hydroxybenzoate | 0.20 |
| Chlorphenesin | 0.27 |
| Nanometric material according to the | 4.00 |
| invention obtained according to example 1B | |

### EXAMPLE 3

### Suncream

| | |
|---|---|
| Pentacyclomethicone | 40.65 |
| Lauryl methicone copolyol | 3.00 |
| Phenyltrimethylpolysiloxane | 7.50 |
| Octyl methoxycinnamate (UV screening agent) | 10.00 |
| Mixed phenoxyethanol/paraben | 0.60 |
| DL-α-Tocopherol acetate | 0.50 |
| Water | 18.00 |
| 1,3-Butylene glycol | 5.00 |
| Nanometric material according to the | 14.75 |
| Invention obtained according to example 1C | |

### EXAMPLE 4

### Day Care Cream in the Form of an Oil-in-Water Emulsion

| Phase A: | |
|---|---|
| Water | 57.5 |
| Methylparaben | 0.1 |
| Chlorphenesin | 0.3 |
| Phenoxyethanol | 0.4 |
| Acrylates/C10-30 alkyl acrylate | 0.5 |
| Crosspolymers | |
| Glycerol | 3.0 |
| 1,3-Butylene glycol | 3.0 |
| Nanometric material according to the | 10.0 |
| invention obtained according to example 1E | |
| Phase B: | |
| Steareth-2 | 1.3 |
| Steareth-21 | 2.2 |
| Glyceryl stearate | 1.0 |
| Cetyl alcohol | 2.2 |
| Stearyl alcohol | 2.2 |
| Stearin 50/50 | 1.8 |
| Cetyl palmitate | 1.3 |
| Hydrogenated polyisobutene | 12.3 |
| Preservatives | 0.7 |
| Phase C: | |
| DL-α-Tocopherol acetate | 0.2 |

Notes:
The “Acrylates/C10-30 Alkyl Acrylate Crosspolymers” are copolymers formed of a C₁₀-C₃₀ alkyl acrylate with one or more acrylic acid, methacrylic acid, acrylic ester or methacrylic ester monomers, crosslinked with a sucrose allyl ether or a pentaerythritol allyl ether. Such copolymers are in particular available commercially from Noveon Inc., USA.
Stearin 50/50 is a 50:50 mixture by weight of hexadecanoic acid and octadecanoic acid. The “preservatives” which appear in the above phase B are composed of a mixture of methyl, ethyl, propyl, butyl and isobutyl esters commercially available from Clariant Corp., USA.

1-18. (canceled)

19. A cosmetic composition comprising at least one cosmetic agent, wherein said composition comprises, as one of said at least one cosmetic agent, at least one nanometric material comprising nitrogen-doped titanium oxides in an amount cosmetically effective for protecting against ultraviolet radiation, alone or in combination with one or more other organic and/or inorganic sunscreens, in a cosmetically acceptable excipient.

20. The composition of claim 19, comprising from 0.1 to 50% by weight of said nanometric material, with respect to the total weight of the composition.

21. The composition of claim 19, comprising from 1 to 15% by weight of said nanometric material, with respect to the total weight of the composition.

22. The composition of claim 19, wherein the nanometric material comprises at least 0.4% of nitrogen atoms, with respect to the total atomic composition of the nanometric material, and between 0 and 40% by weight of carbon, with respect to the total weight of the nanometric material.

23. The composition of claim 19, wherein the nanometric material comprises between 0.5 and 10% of nitrogen atoms and between 0 and 40% by weight of carbon, with respect to the total weight of the nanometric material.

24. The composition of claim 19, wherein said nanometric material has a mean diameter of between 2 nm and 100 nm.

25. The composition of claim 19, wherein said nanometric material has a mean diameter of between 6 nm and 30 nm, in the elementary or agglomerated form.

26. The composition of claim 19, formulated in a cosmetic form selected from the group consisting of a cream, an oil, a gel, a spray, a lotion and a powder.

27. The composition of claim 19, wherein said nanometric material has undergone a surface treatment chosen from:
a) a photostabilization treatment; and
b) at least one treatment for coating with at least one coating layer.

28. The composition of claim 27, wherein:
a) said photostabilization treatment is performed with phosphate anions; and
b) said at least one coating layer is selected from an alumina coating layer and a silica coating layer.

29. A method of cosmetic care for protecting a skin of a person in need thereof against ultraviolet radiation, comprising applying on skin zones in need thereof, a cosmetic composition as defined in claim 19.

30. A method as claimed in claim 29, wherein the nanometric material is obtained by injection of a titanium oxide precursor in liquid or gaseous form and of a nitrogenous compound into a laser pyrolysis reactor.

31. A method as claimed in claim 30, wherein the precursor comprises titanium tetraisopropoxide (TTIP), or titanium tetrachloride (TiCl₄), within an oxygen stream.

32. A method as claimed in claim 30, wherein the nitrogenous compound comprises ammonia NH₃.

33. A method as claimed in claim 30, wherein the flow rate of the nitrogenous compound is between 10 and 2000 cm³ per minute.

34. A method as claimed in claim 30, wherein the precursor in liquid or gaseous state is entrained by a neutral or carrier gas into said reactor, at a flow rate of between 200 cm³ per minute and 4000 cm³ per minute.

35. A method as claimed in claim 30, wherein the precursor in liquid or gaseous state is entrained by a neutral or carrier gas into said reactor, at a flow rate of between 500 and 2000 cm³ per minute.

36. A method as claimed in claim 34, wherein the neutral or carrier gas comprises a sensitizing additive having the role of increasing the absorption of laser energy.

37. A method as claimed in claim 30, wherein the flow rate of the precursor is less than or equal to 1000 g per hour to continually produce nanometric material based on nitrogen-doped titanium oxide comprising between 0 and 40% by weight of carbon, with respect to the weight of the nanometric material.

38. A method as claimed in claim 30, wherein the flow rate of the precursor is about 100 g per hour.

39. A method as claimed in claim 30, wherein, when said material obtained after laser pyrolysis comprises carbon, said material being subjected to an additional heat treatment which removes the carbon to obtain a nanometric material comprising nitrogen-doped titanium oxide.

40. A method as claimed in claim 30, wherein the laser power is between 500 and 2500 watts.

41. A method as claimed in claim 29, wherein said material has undergone a surface treatment chosen from:
a) a photostabilization treatment; and
b) at least one treatment for coating with at least one coating layer.

42. The method of claim 41, wherein:
a) said photostabilization treatment is performed with phosphate anions; and
b) said at least one coating layer is selected from an alumina coating layer and a silica coating layer.
